(19) United States
(12) Patent Application Publication
(10) Pub. No.: US 2010/0191225 A1
(43) Pub. Date: Jul. 29, 2010

(54) SURGICAL INSTRUMENT HANDLE

(76) Inventor: Robert Leonard, Audobon, PA (US)

Correspondence Address: CareFusion Corp./BHGL, P.O. Box 10395, Chicago, IL, 60610, US

(21) Appl. No.: 12/757,706

(22) Filed: Apr. 9, 2010

Related U.S. Application Data
(63) Continuation of application No. 11/186,627, filed on Jul. 21, 2005.
(60) Provisional application No. 60/590,095, filed on Jul. 21, 2004.

(51) Int. Cl. A61B 17/00

(52) U.S. Cl. 606/1

(57) ABSTRACT

A surgical instrument handle including a base, a lever, and an actuator rod. The base defines opposed upper and lower faces, a proximal region, a distal region, an intermediate region, and a bend along the lower face at a transition from the intermediate region to the distal region. The lever defines a leading end, a trailing end, and opposed first and second major faces. The lever and the base are juxtaposed relative to each other, with the trailing end of the lever being pivotally connected to the proximal region of the base such that the lever pivots between an open position and a closed position. The rod includes a first end connected to the lever and a second end distal the base. With this configuration, a user grips the handle in a pistol grip-like fashion, with the bend promoting ergonomically correct handling.

### CROSS-REFERENCE TO RELATED APPLICATIONS

This application claims the benefit of U.S. patent application Ser. No. 11/186,627, filed Jul. 21, 2005, now pending, which claims priority to U.S. Provisional Patent Application No. 60/590,095, filed Jul. 21, 2004, each of which is hereby incorporated by reference in its entirety.

### BACKGROUND

The present invention relates to the field of hand-held devices used in surgical procedures. More particularly, it relates to an improved ergonomic handle design for surgical instruments, such as those used in laparoscopic and endoscopic surgeries.

A variety of surgical procedures require the use of specialized hand-held surgical instruments having articulating tips (i.e., forceps-like devices having movable jaws such as needleholders, graspers, dissectors, etc.). In general terms, such surgical instruments consist of a tool, having an elongated shaft and the articulating tip, along with a handle adapted to control articulation of the tip (e.g., opening and closing of the jaws). This configuration affords a surgeon the ability to adeptly control movement of the tip via the handle, with the tip being located, for many applications, a relatively substantial distance away from the handle (and thus the surgeon). Such surgical instruments are commonly used in laparoscopic and endoscopic procedures, with the articulating tip being employed to perform gripping, grasping, cutting, etc., operations. Regardless, the handle typically includes one or two lever arms that are moveable about a pivot point, with pivoting of the lever arm(s) effectuating movement of the articulating tip.

One conventional surgical instrument handle design of the type described above positions the handle pivot point distal or forward of the user's hand when naturally held. One example of this configuration is the conventional “scissors”-type handle. These scissors-type handles have finger and thumb rings located at the free end of the lever arms that provide a means for receiving force and balancing the instrument when both opening and closing the lever arms of the handle.

A second example of a forward pivot point configuration is a “pliers”-type handle that is functionally similar to the scissors handle, but does not include finger rings. With this approach, movement of the levers from the open to the closed positions is accomplished by closing the palm of the user's hand in a squeezing motion. A bow spring or other spring configuration located between the lever arms is sometimes included as a means to bias the handle in an open position to compensate for the lack of finger rings.

While well-accepted, the above-described forward pivot point-type surgical instrument handles may not coincide with the natural pivot point of the human hand. In recognition of this potential drawback, other surgical handle designs for forceps-like surgical instruments have been devised that incorporate a rearwardly located pivot point. More particularly, the pivot point between the lever arm(s) of the handle is positioned “behind” the user's fingers/hand when naturally held. In other words, as compared to forward pivot point handles with which the pivot point is located between the hand grip surface(s) of the handle and the tip, a rearward pivot point handle locates the hand grip surface(s) between the pivot point and the tip. One such design is described in U.S. Pat. No. 5,498,256, the teachings of which are incorporated herein by reference. This design provides the user with increased leverage and sensitivity when squeezing the lever arm(s), and permits the surgeon to transfer force in a direct linear relationship from the hand to the articulating tip.

Though the rearward pivot point handle designs represent a distinct improvement, ergonomic concerns may still exist. For example, the linear orientation of the handle's lever arm(s) relative to the shaft/articulating tip may not be optimal from an ergonomic standpoint. That is to say, the surgeon may be required to maintain his or her wrist and/or elbow in an uncomfortable position while using the surgical instrument for potentially extended lengths of time. Thus, a need exists for a surgical instrument handle providing rearward pivot control along with enhanced ergonomic comfort.

### SUMMARY

Aspects of the present invention relate to a handle for use as part of a surgical instrument otherwise including a tool having at least one articulating member. The handle includes an elongated base, an elongated lever, and an actuator rod. The base defines opposed upper and lower faces, a proximal region, a distal region, and an intermediate region. In this regard, the base defines a bend along the lower surface thereof at a transition from the intermediate region to the distal region. The lever defines a leading end, a trailing end, and opposed first and second major faces. The lever and the base are juxtaposed relative to each other, with the trailing end of the lever being pivotally connected to the proximal region of the base such that the lever pivots between an open position and a closed position. Finally, the rod includes a first end connectable to the articulating member of the tool and a second end connected to the lever such that the first end is distal the base. With this configuration, a user grips the handle for manipulation of the tool in an ergonomically correct orientation via the bend. To this end, in one embodiment, the bend defines a bend angle of more than 90° and less than 180°; and in another embodiment, the bend angle is in the range of 120°-170°. In another embodiment, the second end of the rod is directly connected to the lever. In yet another embodiment, a link and ball-and-slot assembly facilitates connection of the rod to the lever.

Other aspects of the present invention relate to a surgical instrument including the handle described above and a tool including at least one articulating member. In some embodiments the tool is permanently connected to the handle, and in other embodiments the tool is removably attached to the handle. Regardless, the surgical instrument minimizes strain on the user's wrist during use. In some embodiments, the surgical instrument is configured to allow selective rotation of the articulating member relative to the handle, with the handle incorporating a low-profile linkage assembly that facilitates implementation of a desired bend angle.

Yet other aspects of the present invention relate to a method of using a surgical instrument in various procedures, such as endoscopic or laparoscopic procedures. The method includes providing a surgical instrument including a handle and a tool terminating at a distal tip having at least one articulating member. The handle includes an elongated base, an elongated lever, and an actuator rod. The base defines upper and lower faces, and proximal, distal, and intermediate regions. Further, the base forms a bend along the lower face thereof at a transition of the intermediate region to the distal region. The lever defines first and second opposed major faces, and a trailing end. The trailing end is pivotally connected to the proximal region of the base so as to pivot between an open position and a closed position. The actuator rod includes a first end connected to the tip and a second end connected to the lever. The handle is grasped within a hand of a user such that the index finger of the user's hand is located at or adjacent the distal region of the base along the lower surface thereof and the lever is within a palm of the user's hand. The lever is moved toward the closed position by squeezing the user's hand such that the lever pivots relative to the base at a pivot point rearward of the user's index finger. The handle is maneuvered to position the tip at a desired surgical site located distal the distal end of the base. The tip is maintained at the surgical site while holding the handle, characterized by a wrist of the user being in a neutral position. In some embodiments, the method further includes rotating the tip relative to the handle.

### BRIEF DESCRIPTION OF THE DRAWINGS

FIG. 1 is a segmented, perspective view of a surgical instrument incorporating one embodiment of a handle in accordance with principles of the present invention;

FIG. 2 is a partial cross-sectional view of the handle of FIG. 1;

FIG. 3 schematically illustrates use of the instrument of FIG. 1;

FIG. 4 is a partial cross-sectional view of another embodiment handle in accordance with principles of the present invention;

FIG. 5A is an enlarged side view of a link component associated with the handle of FIG. 4;

FIG. 5B is an enlarged perspective view of a band component associated with the handle of FIG. 4;

FIG. 6 is an exploded view of a rotational assembly associated with the handle of FIG. 4;

FIG. 7 is a partial cross-sectional view of another embodiment handle in accordance with principles of the present invention;

FIG. 8 is an exploded view of a rotational assembly associated with the handle of FIG. 7; and

FIG. 9 is a top, partially segmented view of a portion of the handle of FIG. 7.

### DETAILED DESCRIPTION

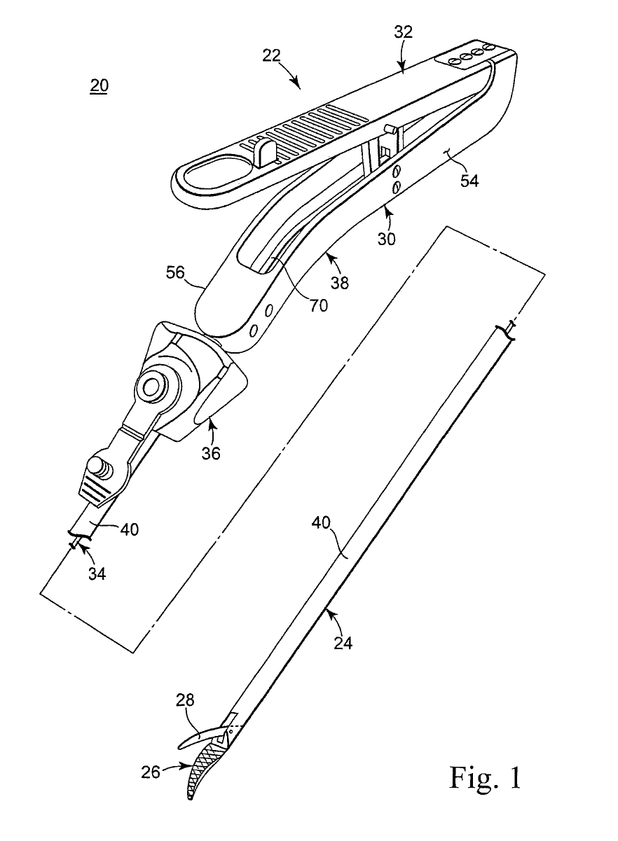

A surgical instrument 20 incorporating one embodiment of a handle 22 in accordance with aspects of the present invention is shown in FIG. 1. The handle 22 is connected to a tool 24 having a tip 26 including at least one articulating member 28. The handle 22 is adapted to effectuate movement of the articulating member(s) 28 and generally includes an elongated base 30, an elongated lever 32, and an actuator rod or cable 34. In some embodiments, the handle 22 further includes a flush port assembly 36. Details on the various components are provided below. In general terms, however, the lever 32 is connected to the actuator rod or cable 34 and pivotally connected to the base 30 such that pivoting of the lever 32 relative to the base 30 causes movement of the rod or cable 34 that in turn moves the articulating member(s) 28 during use. In this regard, the base 30 defines a bend 38 (referenced generally) that otherwise promotes ergonomically correct grasping of the handle 22 by a user (not shown).

The handle 22 can be employed with a wide variety of different tool configurations having various articulating member(s) 28 designs. For example, while the tip 26 is shown in FIG. 1 as providing one articulating member 28 (useful, for example, for holding a surgical needle), two or more articulating members 28 can be provided (such as with a surgical clamp). In addition, the tool 24 can assume a variety of forms and in some embodiments includes a hollow shaft 40 maintaining the tip 26. The shaft 40 can be rigid, with a linear shape or one or more bends. Alternatively, the shaft 40 can be formed of a malleable material to facilitate selective bending by a user, as described, for example, in U.S. Pat. No. 6,139,563 (Cosgrove III, et al.), the teachings of which are incorporated herein by reference. Further, and as described in greater detail below, the instrument 20 can be configured such that the tool 24 is permanently attached to the handle 22 as shown in FIG. 1; in other embodiments, the tool 24 is selectively assembled to, and disassembled from, the handle 22.

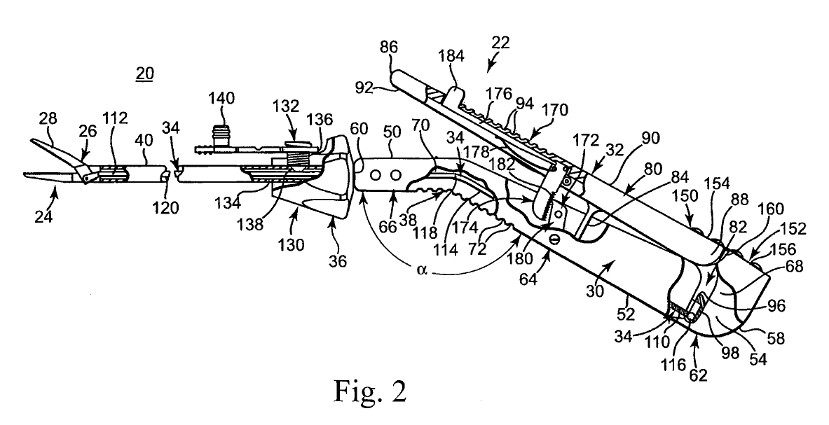

Regardless of an exact configuration of the tool 24, one embodiment of the handle 22 is shown in greater detail in FIG. 2. With reference to the orientation of FIG. 2, the base 30 defines opposing, upper and lower faces 50, 52; opposing, first and second sides 54, 56 (best shown in FIG. 1); a proximal end 58; and a distal end 60. Further, the base 30 can be characterized as defining a proximal region 62, an intermediate region 64, and a distal region 66. The proximal region 62 terminates at the proximal end 58, and, in one embodiment, forms an arm 68. The intermediate region 64 extends between the proximal region 62 and the distal region 66. Finally, the distal region 66 terminates at the distal end 60. In one embodiment, the base 30 defines a slot 70 (referenced generally in FIG. 2 and best shown in FIG. 1) within which various components are received.

In one embodiment, the base 30 is similar to that described in U.S. Pat. No. 5,498,256, the teachings of which are incorporated herein by reference, and is adapted to provide a rearward pivot point relative to the lever 32 via the arm 68 as described below. With this configuration, the lower face 52 of the base 30 serves as a grasping surface for a user's fingers (not shown), when the handle 22 is held in a pistol grip-like fashion. By way of reference, the user's index finger will naturally be positioned along the lower face 52 at or adjacent the distal region 66, and the remaining fingers positioned proximal the index finger. To this end, one or more grip ridges 72 are optionally formed along the lower face 52 as shown. Regardless, as explained below, the base 30 is configured to facilitate a more ergonomically-correct positioning of the user's hand and wrist when the handle 22 is naturally gripped.

As a point of reference, FIG. 2 illustrates the shaft 40 of the tool 24 as extending distally relative to the distal end 60 of the base 30. In one embodiment, the shaft 40 has a substantially straight or linear extension relative to the base 30, with the so-defined longitudinal axis of the shaft 40 serving as a plane of extension of the tip 26 relative to the handle 22, and thus relative to the user's hand. With this in mind, the distal region 66, and in particular the lower face 52 along the distal region 66, defines a plane that is substantially co-planar with the longitudinal axis of the shaft 40. To facilitate ergonomically-correct handling, the intermediate region 64, and in particular the lower face 52 along the intermediate region 64, extends at an angle relative to the distal region 66. More particularly, the bend 38 is formed at the intersection or transition between a plane defined by the lower face 52 along the distal region 66 and a plane defined by the lower face 52 along the intermediate region 64, with the bend 38 forming a bend angle α. The bend 38 can be formed as a distinct angle, or as a curve. Regardless, in one embodiment, the bend angle α is greater than 90° and less than 180°. In another embodiment, the bend angle α is in the range of 120°-170°; more preferably in the range of 145°-155°; even more preferably in the range of 147°-153°. It has surprisingly been found that for surgical tools having an elongated shaft and articulating tip, a bend angle α of 150°.+−0.3° provides the greatest level of ergonomic comfort, although other bend angles described above are also acceptable. The base 30 can be formed from a variety of surgically compatible materials, such as stainless steel, plastic, etc. In one embodiment, the base 30 is integrally formed such that the bend 38 is permanently maintained and cannot be altered by a user.

The lever 32 includes a lever body 80 and a leg 82. In one embodiment, the lever 32 further includes a spacer 84 adapted to prevent overt rotation of the lever body 80 relative to the base 30. Regardless, the lever body 80 defines a leading end 86, a trailing end 88, and first and second opposing faces 90, 92. The lever body 80 can assume a variety of forms, and in one embodiment, is assembled relative to the base 30 such that the second face 92 is adjacent the upper face 50, with the first face 90 providing a surface for grasping within a user's palm (not shown). To this end, the first face 90 optionally forms one or more grip ridges 94. Regardless, the leg 82 extends downwardly (relative to the orientation of FIG. 2) from the trailing end 88 of the lever body 80, and is adapted to facilitate mechanical connection with the actuator rod 34. For example, in one embodiment, the leg 82 forms a groove 96 (shown generally) at a leading portion 98 thereof within which a corresponding component associated with the actuator rod 34 is received, as described below. Alternatively, coupling with the actuator rod 34 can be accomplished in a variety of other fashions. Preferably, however, the leg 82 is configured such that the leading portion 98 moves with pivoting motion of the lever body 80 relative to the base 30.

The actuator rod or cable 34 is configured to provide a mechanical link between the lever 32 and the articulating member(s) 28 (FIG. 1), and thus can assume a variety of forms. As used throughout this specification, reference to an “actuator rod” is intended to encompass both rigid and flexible bodies, such that an “actuator rod” is inclusive of a “cable”. With the one embodiment of FIG. 2, for example, the actuator rod 34 directly connects the lever 32 and the articulating member(s) 28, defining a first or proximal end 110, a second or distal end 112, and an intermediate segment 114. The proximal end 110 is configured for coupling with the leading portion 98 of the leg 82 and thus, in one embodiment, includes a sphere or ball-like body 116 sized to be captured within the groove 96 formed by the leg 82. Alternatively, the proximal end 10 can incorporate other features corresponding with the mounting feature provided by the leg 82. The distal end 112 is similarly adapted for coupling to a corresponding element of the articulating member(s) 28. Regardless, the intermediate segment 114 is adapted to be received within the slot 70 of the base 30. With this in mind, in one embodiment, the intermediate segment 114 forms a curve 118 commensurate with the bend 38 defined by the base 30.

As described below, in alternative embodiments, the actuator rod 34 is indirectly connected to the lever 32, and thus can assume a variety of other forms. In addition, for embodiments in which the tool 24 incorporates a malleable material for the shaft 40, at least a distal segment 120 of the rod 34 (i.e., that portion of the rod 34 positioned distal the base 30) is also formed of a malleable material such that the rod 34 will bend with bending of the shaft 40. In this regard, the actuator rod 34 can be unitarily or homogenously formed of a malleable material; alternatively, the rod 34 can consist of two or more discretely formed and subsequently assembled (e.g., welding, adhesive, mechanical coupling, etc.) to one another. As previously described, the actuator rod 34 can be a rigid body or a more flexible body such as a cable.

The flush port assembly 36 is optionally included with one embodiment of the present invention, and generally includes a collar or knob 130, a Luer lock 132, and an adapter tube that, with the one embodiment of FIG. 2, is provided as a portion of the shaft 40. The flush port assembly 36 is, in one embodiment, akin to that described in U.S. Pat. No. 5,489,290, the teachings of which are incorporated herein by reference. In general terms, the collar 130 is configured to be assembled over the shaft 40 and defines a longitudinal passage 134 and a transverse opening 136. The transverse opening 136 is configured and sized to receive the Luer lock 132 and is fluidly connected to the longitudinal passage 134. The longitudinal passage 134 in turn, is sized to coaxially receive the shaft 40 that otherwise forms a port 138 fluidly connected to an internal lumen thereof. Upon final assembly, the Luer lock 132 is fluidly connected to the port 138 such that fluid can be delivered to the internal lumen of the shaft 40 via the Luer lock 132. In one embodiment, a plug 140 is provided for selectively closing the Luer lock 132. To minimize backflow of fluids otherwise entering the lumen of the shaft 40 via the Luer lock 132, a seal (not shown) can be provided at a proximal end of the shaft 40, otherwise fluidly sealing the shaft 40 relative to the rod 34 (or other component) upon final assembly. Notably, the flush port assembly 36 is optional such that in alternative embodiments, the flush port assembly 36 can be eliminated from the handle 22.

In general terms, the handle 22 can be assembled by mounting the flush port 36 relative to the shaft 40. The actuator rod 34 is coaxially positioned within the shaft 40, with the distal end 112 thereof being connected to the articulating member(s) 28. The actuator rod 34 is assembled to the base 30 such that the flush port 36 is distally adjacent the distal end 60 of the base 30, and the intermediate segment 114 of the rod 34 is positioned within the slot 70 formed by the base 30. As shown in FIG. 2, the curve 118 formed by the rod 34 is generally aligned with the bend 38 formed by the base 30. The leg 82 of the lever 32 is connected (e.g., mounted) to the proximal end 110 of the rod 34. Finally, the trailing end 88 of the lever body 80 is pivotally mounted to the arm 68 of the base 30.

In one embodiment, a connecting means 150 (referenced generally) is employed to assemble the base 30/lever 32. The connecting means 150 connects the trailing end 88 of the lever body 80 to the proximal region 62 of the base 30 in a manner that allows the lever 32 to pivot about a horizontal axis between an open position (shown in FIG. 2) and a closed position (shown in FIG. 1). In one embodiment, the connecting means 150 is a leaf spring 152 having a pre-selected thickness with a first end 154 and an opposite second end 156. The first end 154 of the spring 152 is connected to the first face 90 of the lever body 80 adjacent the trailing end 88, and the second end 156 of the spring 152 is connected to the arm 68 of the base 30. In addition, the leaf spring 152 may also function as a biasing means for normally biasing the lever 32 to the open position relative to the base 30. In an alternative embodiment, the leaf spring 152 is removably connected to the lever 32 and the base 30 such that the leaf spring 152 may interchangeably be comprised of one of a plurality of leaf springs of different pre-selected thicknesses, thereby varying the resistance of the biasing means. Alternatively, hinged or pivotable attachment of the lever 32 to the base 30 can be accomplished with a variety of other components, such as a screw or other mechanical fastener to name but a few. Regardless, upon final assembly, a rearward pivot point 160 is established between the base 30 and the lever 32, with the second face 92 of the lever body 80 positioned adjacent the upper face 50 of the base 30 such that the base 30 and the lever 32 are juxtaposed to each other relative to a length of the lever body 80 and a length of the intermediate region 64 of the base 30. Further, transitioning of the lever 32 from the open position to the closed position pulls or displaces (via the leg 82) the proximal end 110 of the actuator rod 34 proximally (i.e., toward the proximal end 58 of the base 30), thus causing movement of the articulating member(s) 28.

In one embodiment, to secure the lever 32 relative to the base 30 at selected points throughout a range of motion of the lever 32 between the open and closed positions, a ratchet mechanism 170 (referenced generally) is provided. The ratchet mechanism 170 includes, in one embodiment, a pawl plate 172, a ratchet arm 174, a ratchet lever 176, and a biasing device 178. The pawl plate 172 forms a toothed surface 180 and is attached to the base 30. The ratchet arm 174 similarly includes a toothed surface 182 and is pivotally connected to the lever body 80.

The ratchet lever 176 is also movably connected to the lever body 80 and the ratchet arm 174, and further interfaces with the ratchet arm 174 via the biasing device 178. In this regard, an actuator 184 is provided at a terminal end of the ratchet lever 176 and projects beyond an exterior of the lever body 80 for interface with the user's hand/fingers (not shown). The ratchet lever 176 is mounted to the lever body 80 so as to bias the ratchet arm 174, via the biasing device 178, toward a naturally engaged position whereby the toothed surface 182 of the ratchet arm 174 is oriented to engage the toothed surface 180 of the pawl plate 172. For example, in one embodiment, the biasing device 178 is a thin leaf spring; alternatively, other components (e.g., a torsions spring) can be included with the ratchet mechanism 170 interconnecting the ratchet arm 174/ratchet lever 176 such that in a normal state, the ratchet lever 176 biases the ratchet arm 174 to the engaged position. The ratchet arm 174 can be released from the pawl plate 172 by pressing the actuator 184; assembly of the ratchet lever 176 to the ratchet arm 174 is such that when the actuator 184 is depressed, the ratchet lever 176 draws the ratchet arm 174 away from the pawl plate 172. Alternatively, the ratchet mechanism 170 can assume a wide variety of other forms. Even further, in alternative embodiments, the ratchet mechanism 170 is eliminated entirely.

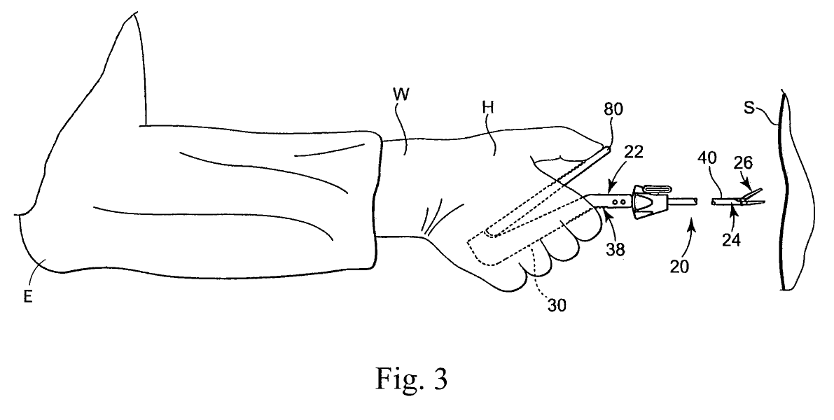

During use, the instrument 20 is provided to the user as an integral structure, with the handle 22 being permanently mounted to the tool 24. The handle 22 is grasped within a hand of the user such that the lever body 80 is within the user's palm, and the user's fingers wrap about the base 30, contacting or grasping the lower face 52 thereof. One preferred handling technique entails the user's index finger being at or adjacent the distal end 60 of the base 30 (at the lower face 52 thereof), and thus opposite the pivot point 160 established between the base 30 and the lever 32. In other words, the handle 22 represents a rearward pivot point-type design. The user's thumb wraps about or contacts the first face 90 of the lever body 80 at or adjacent the leading end 86 thereof. As schematically shown in FIG. 3, the tip 26 is then directed toward a surgical site S by maneuvering the handle 22. A grasping operation can be performed (either before, after, and/or simultaneously with positioning of the tip 26 at the surgical site S) in which the articulating member(s) 28 of the tip 26 is caused to move or articulate by squeezing the user's hand H to force the lever body 80 toward the base 30 (i.e., transitioning from the open position to the closed position), or vice-versa. Regardless, the bend 38 defined by the base 30 allows the user to maintain his or her wrist W in an ulnar neutral position relative to the hand H otherwise grasping the handle 22 while otherwise maintaining the tip 26 at the surgical site S (and/or moving the tip 26 relative to the surgical site S, such as when applying a needle to or through tissue at the surgical site S). Thus, the handle 22 facilitates a neutral wrist position via an ergonomically optimal angle relative to the tool 24 when the handle 22 is held in a pistol grip-like fashion by the user's hand H. The user's forearm is thus generally aligned with the shaft 40, and eliminates the need for the user to “raise” his or her elbow E during use. This represents a distinct improvement over previous configurations.

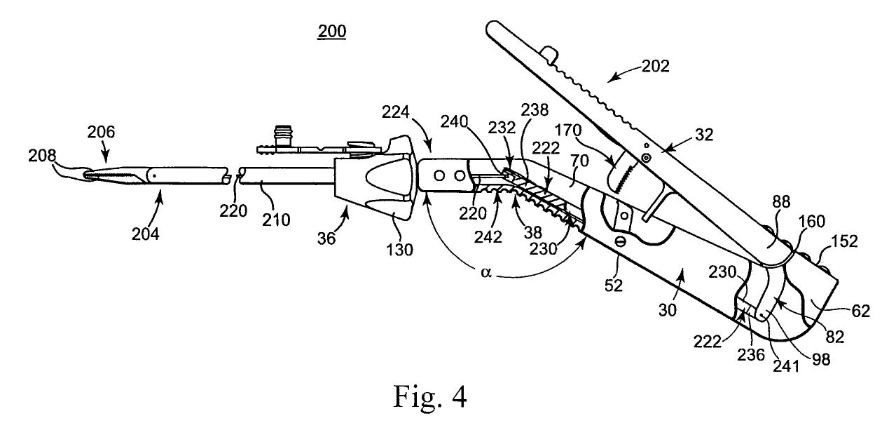

An alternative embodiment surgical instrument 200 is shown in FIG. 4, and generally includes a handle 202 and a tool 204. In many respects, the surgical instrument 200 is highly similar to the surgical instrument 20 (FIG. 1) previously described, with the tool 204 including a tip 206 having at least one articulating member 208 and a shaft 210. The handle 202 is mounted to the tool 204 and is adapted to facilitate movement or articulation of the articulating member(s) 208. In addition, the handle 202 is configured to allow rotation of the tool 204 relative to the handle 202 as described below.

In one embodiment, the handle 202 is highly similar to the handle 20 (FIG. 2) previously described, and includes the base 30, the lever 32, and (optionally) the flush port assembly 36. The trailing end 88 of the lever 32 is pivotally or hingedly mounted to the proximal region 62 of the base 30 via the leaf spring 152 (or other connecting means), thus defining the rearward pivot point 160. Further, the handle 202 optionally includes the ratchet mechanism 170 previously described. Once again, the base 30, and in particular the lower face 52 thereof, forms the bend 38 having the bend angle α as previously described. In addition, the handle 202 includes an actuator rod 220, a linkage assembly 222, and a rotational assembly 224 (referenced generally). In general terms, the linkage assembly 222 connects the actuator rod 220 with the lever 32. The rotational assembly 224 is configured to allow the tool 204 to rotate relative to the base 30.

In one embodiment, the linkage assembly 222 has a low profile and includes a link 230 and a band 232. The link 230 defines a proximal portion 234 terminating at a proximal end 236 and a distal portion 238 terminating at a distal end 240. The proximal portion 234 is configured to facilitate rotatable or pivotable mounting to the leading portion 98 of the leg 82 otherwise provided as part of the lever 32. For example, in one embodiment, the proximal portion 234 and the leg 82 are configured to be attached to one another using a pin 241. Alternatively, a variety of other mounting techniques can be employed, such as, for example, ball-and-socket.

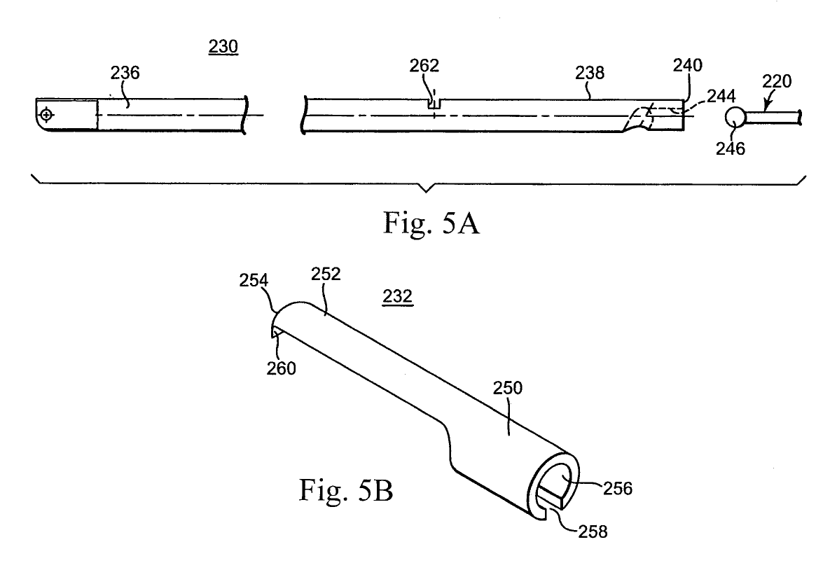

The distal portion 238 of the link 230 is configured for connecting with the rod 220. For example, in one embodiment, the link 230 and the actuator rod 220 employ a ball-and-slot assembly 242. In one embodiment, and with additional reference to FIG. 5A, the link 230 forms a slot 244 at the distal portion 238 thereof, extending to the distal end 240. The slot 244 is thus transversely and longitudinally open relative to the distal end 240 and a portion of the longitudinal length of the distal portion 238. Conversely, the rod 220 (a portion of which is shown in FIG. 5A) terminates at a ball or sphere-like body 246. The slot 244 is sized to receive the ball 246, as well as to provide clearance about a portion of the actuator rod 220 otherwise extending from the ball 246. In one embodiment, the ball 246 is captured within the slot 244 via the band 232.

One embodiment of the band 232 is shown in FIG. 5B. The band 232 has a generally tubular shape, including a distal segment 250 and a proximal segment 252 terminating at a proximal end 254. As compared to the proximal segment 252, the distal segment 250 defines a nearly complete tubular body having an inner surface 256 and defining a longitudinal gap 258. With additional reference to FIG. 5A, the inner surface 256 defines a diameter approximating an outer diameter of the link 230, such that the distal segment 250 frictionally engages the link 230 upon assembly. The distal segment 250 has a longitudinal length slightly longer than that of the slot 244, with the gap 258 having a circumferential width less than a diameter of the ball 246. Thus, when applied over the link 230, the distal segment 250 retains the ball 246 within the slot 244, with the gap 258 providing sufficient clearance for the rod 220 otherwise extending from the ball 246. To promote a more rigid connection between the band 232 and the link 230, the band 232 defines, in one embodiment, a finger 260 at the proximal end 254. The finger 260 extends radially inwardly relative to the proximal segment 252 and is sized to be captured within a corresponding aperture 262 formed along the distal portion 238 of the link 230. Thus, upon final assembly, the band 232 is secured to the link 230 via nesting of the finger 260 within the aperture 262, with the distal segment 250 retaining the ball 246 within the slot 244.

The link 230/band 232 configuration beneficially promotes ease of manufacture, with the band 232 simply sliding over the link 230 until the finger 260 is aligned with, and is thus received in, the aperture 262. Further, the band 232 can easily be disassembled from the link 230 by lifting the finger 260 from the aperture 262 (e.g., with the user's finger/finger nail), thereby allowing a user to rapidly adjust or repair the handle 22 (FIG. 4). Alternatively, the ball-and-slot assembly 242 can be reversed, with the link 230 forming or being connected to a ball and the rod 220 forming or being connected to a component otherwise forming a slot. Regardless, the ball-and-slot assembly 242, and in particular the band 232, establishes a low profile so as to not interfere with desired movement of the lever 32 (FIG. 4) during use. Alternatively, other configurations can be employed to capture the ball 246 within the slot 244, such as set screws, welding, etc. Similarly, while the finger 260 has been described as facilitating fixation of the band 232 relative to the link 230, other assembly techniques, such as screws, welding, adhesives, crimping, etc., can also be employed.

Regardless of an exact configuration, upon final assembly and with specific reference to FIG. 4, the linkage assembly 222 connects the actuator rod 220 to the lever 32 such that the actuator rod 220 is linearly oriented (e.g., coaxially aligned with the shaft 210) and can be rotated relative to the base 30 (and the handle 32) in conjunction with the rotational assembly 224 as described below. Further, the linkage assembly 222 provides requisite connection between the lever 32 and the actuator rod 220 in a low profile manner whereby an entirety of the linkage assembly 222 (as well as the corresponding segment of the rod 220) nests within the slot 70 (referenced generally; best shown in FIG. 1) formed by the base 30 to accommodate the bend angle α. In other words, the linkage assembly 222 coincides with the bend 38 in the base 30 and thus does not interfere with desired movement of the lever 32 to a closed position.

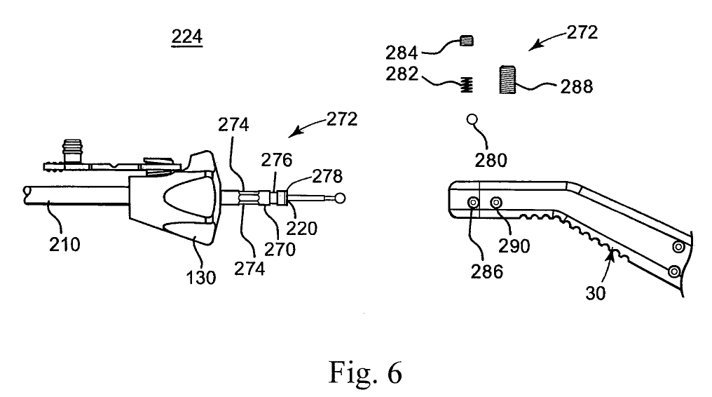

With reference to FIG. 6, in one embodiment, the rotational assembly 224 includes an adaptor tube 270 forming a portion of a detent mechanism 272 (referenced generally). With the one embodiment of FIG. 6, the adaptor tube 270 is provided as an integral portion of the shaft 210; alternatively, the adaptor tube 270 can be separately provided and physically connected with the shaft 210 via the collar 130. Regardless, the adaptor tube 270 is sized to coaxially receive the actuator rod 220 and forms, in one embodiment, a plurality of circumferentially aligned radial grooves 274 and a circumferential slot 276 adjacent a proximal end 278 thereof. The grooves 274 are configured to selectively receive a corresponding component of the detent mechanism 272, whereas the circumferential slot 276 is configured to facilitate assembly of the adaptor tube 270 to the base 30 in a manner allowing rotation of the tube 270 relative to the base 30.

With the above in mind, and in one embodiment, the detent mechanism 272 further includes a ball 280 and a spring 282. The ball 280 is sized to be selectively received within a corresponding one of the grooves 274, with the spring 282 biasing the ball 280 into engagement therewith. In one embodiment, a first set screw 284 secures the ball 280/spring 282 within an opening 286 formed by the base 30 and bears against the spring 282 opposite the ball 280. A second set screw 288 can be provided for retaining the adaptor tube 270 relative to the base 30 in a rotationally-stable manner. In particular, upon final assembly, the set screw 288 is secured to the base 30 via an opening 290 and slidably nests within the circumferential slot 276 of the adaptor tube 270. With this configuration, then, the adaptor tube 270 can rotate relative to the base 30, with the detent mechanism 272 selectively retaining the adaptor tube 270 at a desired rotational position relative to the base 30. The set screw 288/circumferential slot 276 interface prevents longitudinal displacement of the adaptor tube 270 relative to the base 30. In alternative embodiments, the rotational assembly 224 is configured to provide only a minimal frictional resistance to rotation, and thus, for example, may not include the detent mechanism 272. Even further, the rotational assembly 224 can be configured to allow for free rotation.

Returning to FIG. 4, during use, the surgical instrument 200 is grasped and handled in a manner virtually identical to that described with respect to the instrument 20. In particular, the bend 38 formed by the base 30 facilitates an ergonomically-correct positioning of the user's hand and wrist during normal procedures. Where desired, the tip 206 can be rotated relative to the handle 202, and in particular the base 30/lever 32, by, for example, grasping the collar 130 and applying a rotational force. Because the collar 130 is secured to the shaft 210, a rotational force imparted upon the collar 130 is transferred onto the shaft 210 that in turn forces to the tip 206 to rotate. As described above, in one embodiment, the detent mechanism 272 (FIG. 6) selectively frictionally “locks” the shaft 210, and thus the tip 206, at successive rotational positions (via the grooves 274 (FIG. 6)); in alternative embodiments, only a slight frictional resistance to rotation of the shaft 210 relative to the base 30 is provided; and in even further alternative embodiments, the shaft 210 rotates freely relative to the base 30. Regardless, depending upon the manner in which the articulating member(s) 208 is connected to the actuator rod 220, rotation of the tip 206 is translated to the rod 220. The rotational assembly 224 allows for rotation of the rod 220 relative to the link 230, and thus relative to the base 30/lever 32. Importantly, the linkage assembly 222 and the rotational assembly 224 allow for operation of the lever 32 between the open and closed positions as well as rotation of the tip 206 in a manner that allows for the desired bend angle oc.

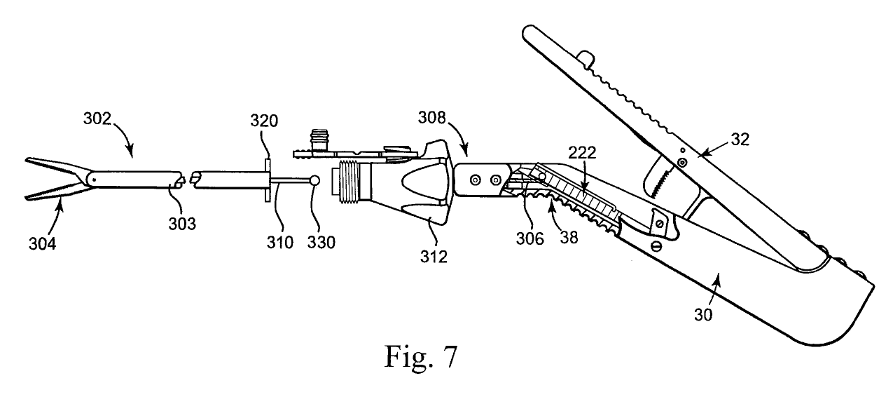

While the surgical instrument 20 (FIG. 1), 200 has been described as incorporating a permanent mounting between the respective handle and tool, in alternative embodiments, the handle can be configured for selective assembly to, and disassembly from, the tool. For example, FIG. 7 illustrates an alternative embodiment handle 300 for use as part of a surgical instrument incorporating a tool 302 (shown generally) otherwise having a shaft 303 and at least one articulating member 304 (akin to the articulating member 208 shown in FIG. 4). The handle 300 is highly similar to the handle 202 (FIG. 4) previously described, and includes the base 30 and the lever 32. The handle 300 further include an actuator rod 306 otherwise connected to the lever 32 via the linkage assembly 222 (referenced generally). The handle 300 further includes a rotational assembly 308 (referenced generally). As described in greater detail below, the actuator rod 306 is not directly coupled to the articulating member 304, but instead is indirectly coupled via a secondary rod 310 associated with the tool 302. In addition, a collar 312 is provided to facilitate releasable assembly with the tool 302.

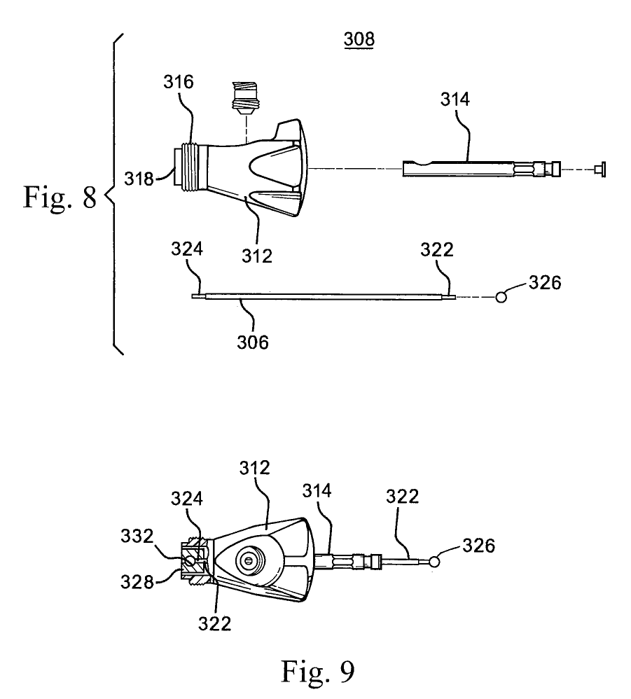

With additional reference to FIG. 8, the rotational assembly 308 includes the collar 312, an adaptor tube 314, and a detent mechanism (not shown but akin to the detent mechanism 272 of FIG. 6). The adaptor tube 314 is similar to the adaptor tube 270 (FIG. 4) previously described and is sized to coaxially receive the actuator rod 306. However, unlike previous embodiments, the adaptor tube 314 is provided apart from the shaft 303 (FIG. 7; otherwise associated with the tool 302). With this in mind, the collar 312 forms a longitudinal passage (not shown) within which the adaptor tube 314 is received. In addition, the collar 312 forms, in one embodiment, external threads 316 adjacent a distal end 318 thereof, with the threads 316 adapted to threadably received a corresponding component 320 (FIG. 7) of the tool 302. The actuator rod 306 has a length slightly greater than that of the adaptor tube 314, defining opposing first and second ends 322, 324. The first end 322 is configured for attachment to the linkage assembly 220 and thus, in one embodiment, forms or is attached to a ball 326. Conversely, the second end 324 is configured for attachment to, or integrally forms, a receptacle 328, as shown in FIG. 9. In general terms, the receptacle 328 can assume a wide variety of forms and is adapted to releasably receive a corresponding element 330 (FIG. 7) associated with the secondary rod 310 (FIG. 7) of the tool 302 (FIG. 7). For example, the receptacle 328 forms a hole 332 sized to receive a ball 330 otherwise provided with the secondary rod 310. Regardless, the receptacle 328 is assembled to the second end 324 of the actuator rod 306 in a variety of fashions. For example, in one embodiment, the second end 324 forms a threaded surface that threadably engages a corresponding surface formed at an interior of the receptacle 328.

Upon final assembly of the handle 300 and with reference to FIGS. 7 and 9, the tool 302 can be selectively mounted to, and released from, the handle 300. For example, the secondary rod 310 is secured to the receptacle 328, thus mechanically coupling the actuator rod 306 to the secondary rod 310, and thus the articulating member(s) 304. The shaft 303 is secured to the collar 312, such as by threaded engagement between the components 312, 320. Once assembled, the resultant instrument can be used in a manner identical to that described with respect to the instrument 200 (FIG. 4), with the bend 38 provided by the base 30 promoting ergonomically correct handling by a user. Once again, the lever 32 is pivoted relative to the base 30 to effectuate movement or articulation of the articulating member(s) 304. Further, the articulating member(s) 304 can be rotated relative to the handle 300 by imparting a rotational force onto the collar 312. The rotational assembly 308 allows the actuator rod 306 to rotate with rotation of the articulating member(s) 304.

In yet another alternative embodiment (not shown), the non-rotating handle 22 design of FIG. 1 can be modified to facilitate removable attachment between the tool 24 and the handle 22, akin to the configuration described with respect to FIG. 7.

The surgical instrument handle of the present invention provides a marked improvement over previous designs. A rearward pivot point is established in conjunction with a bend that promotes ergonomically correct handling. Unlike standard ring handle designs of most laparoscopic instruments, the handle of the present invention enables the surgeon to position the elbows and wrists down by his or her side. This allows the surgeon to perform procedures, such as laparoscopic surgery, in a more natural and comfortable position. The pistol grip design of the handle enhances ease-of-use, minimizes hand fatigue, and increases tactile feedback. In some embodiments, the handle incorporates a low profile linkage assembly that not only accommodates the desired bend, but facilitates rotation of a surgical instrument tool relative to the handle.

Although the present invention has been described with reference to preferred embodiments, workers skilled in the art will recognize that changes can be made in form and detail without departing from the spirit and scope of the present invention.

I claim:

1. A handle for use as part of a surgical instrument including a tool having at least one articulating member, the handle comprising:
an elongated base defining opposed upper and lower faces, opposed first and second sides, a proximal region terminating at a proximal end, a distal region terminating at a distal end, and an intermediate region between the proximal and distal regions, wherein:
the base forms a bend along the lower surface thereof at a transition from the intermediate region to the distal region;
an elongated lever defining a leading end, a trailing end, and opposed first and second major faces, wherein:
the lever and the base are juxtaposed to each other relative to a length of the intermediate region and a length of the lever such that the first major face of the lever is adjacent to and juxtaposed across from the upper face of the base along the intermediate region but not along the leading end of the lever, nor along the distal base region that is distal of the bend, which is angled away from the leading end of the lever,
the trailing end of the lever is pivotally connected to the proximal region of the base such that the lever pivots relative to the proximal region of the base between an open position having a greater space between the first major lever face and the upper face of the base and a closed position having a lesser space between the first major lever face and the upper face of the base;
an actuator rod configured for actuating an articulating member of a tool otherwise mounted to the handle, the rod including a first rod end connected to the lever and a second rod end opposite the first rod end, wherein the rod is assembled such that the second rod end is positioned distal the distal end of the base, wherein the tool is attached to the second rod end and is configured such that the open position and closed position of the lever relative to the base correspond to an open position and closed position of the tool;
a link having a lever-connected portion that is connected to the lever; and
a ball-and-slot assembly connecting the link to the first rod end.

2. The handle of claim 1, wherein the link extends within a slot defined by the base.

3. The handle of claim 1, wherein the ball-and-slot assembly includes: a ball associated with the first end of the rod; and the link forming a slot opposite the lever for receiving the ball.

4. The handle of claim 3, further comprising: a band assembled to the link to capture the ball within the slot.

5. The handle of claim 3, wherein the slot is longitudinally and transversely open relative to the link.

6. The handle of claim 1, wherein the ball-and-slot assembly includes:
the first end of the rod forming a slot; and
a ball associated with the link opposite the lever-connected portion of the link.

7. The handle of claim 1, wherein the link and the rod are substantially linear in longitudinal extension, with the ball-and-slot assembly configured to permit pivoting of the link relative to the rod with movement of the lever.

8. The handle of claim 1, wherein the distal region forms the lower surface to define a first plane adjacent the intermediate region, and the intermediate region forms the lower face to define a second plane adjacent the distal region, and further wherein an intersection of the first and second planes forms the bend to define a bend angle of more than 90° and less than 180°.

9. The handle of claim 8, wherein the bend angle is in the range of 120-170°.

10. The handle of claim 8, wherein the bend angle is in the range of 145°-155°.

11. The handle of claim 1, wherein the base is configured to permanently maintain the bend.

12. The handle of claim 1, further comprising: biasing means for biasing the lever to the open position.

13. The handle of claim 1, further comprising:
a tube extending distally from the distal end of the base and defining a lumen;
a transverse hole in fluid communication with the lumen, the rod being slidably received within the tube;
a collar connected to an exterior of the tube, having a collar opening therethrough that is in registration with the hole in the tube wherein the collar and the tube are rotatable relative to the base; and
a hollow shaft extending through said collar opening and fluidly connected with the lumen.

14. A method of using a surgical instrument, the method comprising:
providing a surgical instrument according to claim 1, further comprising a tool tip operably connected to the handle and actuator rod;
grasping the handle with a hand of a user such that an index finger of the user's hand is located at or adjacent the distal region of the base along the lower surface thereof opposite a pivot point established between the lever and the base, and the lever is within a palm of the user's hand;
moving the lever toward the closed position by squeezing of the user's hand such that the lever pivots relative to the base at a point opposite the user's index finger; maneuvering the handle to position the tip at a desired surgical site located distal the distal end of the base; and
maintaining the tool tip at the surgical site while holding the handle, characterized by: a wrist of the user, otherwise associated with the hand grasping the handle, being held in a neutral position.

15. The method of claim 13, further comprising: rotating the tip relative to the handle with the lever in the closed position.

16. A handle for use as part of a surgical instrument including a tool having at least one articulating member, the handle comprising:
an elongated base defining opposed upper and lower faces, opposed first and second sides, a proximal region terminating at a proximal end, a distal region terminating at a distal end, and an intermediate region between the proximal and distal regions, wherein:
the base forms a bend along the lower surface thereof at a transition from the intermediate region to the distal region;
an elongated lever defining a leading end, a trailing end, and opposed first and second major faces, wherein:
the lever and the base are juxtaposed to each other relative to a length of the intermediate region and a length of the lever such that the first major face of the lever is adjacent to and juxtaposed across from the upper face of the base along the intermediate region but not along the leading end of the lever, nor along the distal base region that is distal of the bend, which is angled away from the leading end of the lever,
the trailing end of the lever is pivotally connected to the proximal region of the base such that the lever pivots relative to the proximal region of the base between an open position having a greater space between the first major lever face and the upper face of the base and a closed position having a lesser space between the first major lever face and the upper face of the base;
a hollow shaft extending distally from the distal end of the base;
an actuator rod extending through the hollow shaft and configured for actuating an articulating member of a tool, the rod including a first rod end connected to the lever and a second rod end opposite the first rod end, wherein the rod is assembled such that the second rod end is positioned distal the distal end of the base,
wherein the tool is attached to the second rod end and to the hollow tube, and is configured such that the open position and closed position of the lever relative to the base correspond to an open position and a closed position of the tool;
wherein the rod extends within the base from the first end and forms a curved segment having a curve commensurate with the bend.

17. The handle of claim 16, wherein the first end of the rod is directly attached to the lever.

18. A surgical instrument comprising:
a handle comprising:
an elongated base defining opposed upper and lower faces, opposed first and second sides, a proximal region terminating at a proximal end, a distal region terminating at a distal end, and
an intermediate region between the proximal and distal regions, wherein the base forms a bend along the lower surface thereof at a transition from the intermediate region to the distal region,
an elongated lever defining a leading end, a trailing end, and opposed first and second major faces, wherein the lever and the base are juxtaposed to each other relative to a length of the intermediate region and a length of the lever such that the first major face of the lever is adjacent the upper face of the base along the intermediate region, but not along the leading end of the lever, nor along the distal base region that is distal of the bend, which is angled away from the leading end of the lever,
wherein the trailing end of the lever is pivotally connected to the proximal region of the base such that the lever pivots relative to the base between an open position and a closed position;
an actuator rod including a first end connected to the lever and a second end extending distal the distal end of the base; and
a tool connected to the handle, the tool comprising:
a tip having at least one articulating member, wherein the articulating member of the tool is connected to the second end of the rod such that movement of the lever between the open and closed positions causes movement of the articulating member.

19. The surgical instrument of claim 18, wherein the tool is removably assembled to the handle.

20. The surgical instrument of claim 18, wherein the bend defines a bend angle in the range of 145°-155°.
